(19) United States
(12) Patent Application Publication
(10) Pub. No.: US 2019/0328590 A1
(43) Pub. Date: Oct. 31, 2019

(54) Disposable Integrated Bag for a Feminine Sanitary Napkin

(71) Applicant: Rochelle Alexandria Johnson, McDonough, GA (US)

(72) Inventor: Rochelle Alexandria Johnson, McDonough, GA (US)

(21) Appl. No.: 16/057,697

(22) Filed: Aug. 7, 2018

Related U.S. Application Data
(60) Provisional application No. 62/762,306, filed on Apr. 30, 2018.

(51) Int. Cl. A61F 13/551; A61F 13/472; A61F 13/514

(52) U.S. Cl. CPC A61F 13/5515; A61F 2013/55155; A61F 13/51478; A61F 13/472

(57) ABSTRACT

An feminine sanitary napkin and/or absorbent pad of oval configuration contains an integrated plastic bag for discreet content disposal, imprinted with a design of solid colors or patterns to present a feminine look, conceal odor, leakage and exposure. The integrated bag is measured, glued, and folded to fit between the 3^{rd} and 4^{th} layers of the absorbent pad. The bag contains an easy grip pull flap at the front tip of the pad, when ready to discard from undergarments, pull flap forward and out to open, and roll to tuck the soiled pad into the bag. The plastic material is made to fit small, medium, large and oversize feminine napkins after each use. In addition, made to reduce contamination on the hands, over usage of tissue, and it will encourage for proper disposal of the content.

### BACKGROUND OF THE INVENTION

The invention of a disposable integrated plastic bag between the assembly of the layers of the absorbent pad, to be used for convenient and considerate disposal of a used feminine sanitary napkin. Materials such as adhesive, plastic, and glue, will be assembled with related items pertaining to a feminine sanitary napkin and/or absorbent pad. The disposable integrated plastic bag is made of related material to block odor, leakage, and exposure, with imprinted colors and/or designs. The dimensions of the absorbent pad is surrounded by the mid-portion to develop an integrated bag folding technique with an opening once pulled on the tip of flap. Ideally, the flap will pull out and open the plastic bag half way facing the user to easily roll the feminine napkin inside for discreet disposal.

### Product Labeling & Descriptions

The invention drawings is shown with a combination of letters and numbers. A list below will assist identifying the components of the invention.

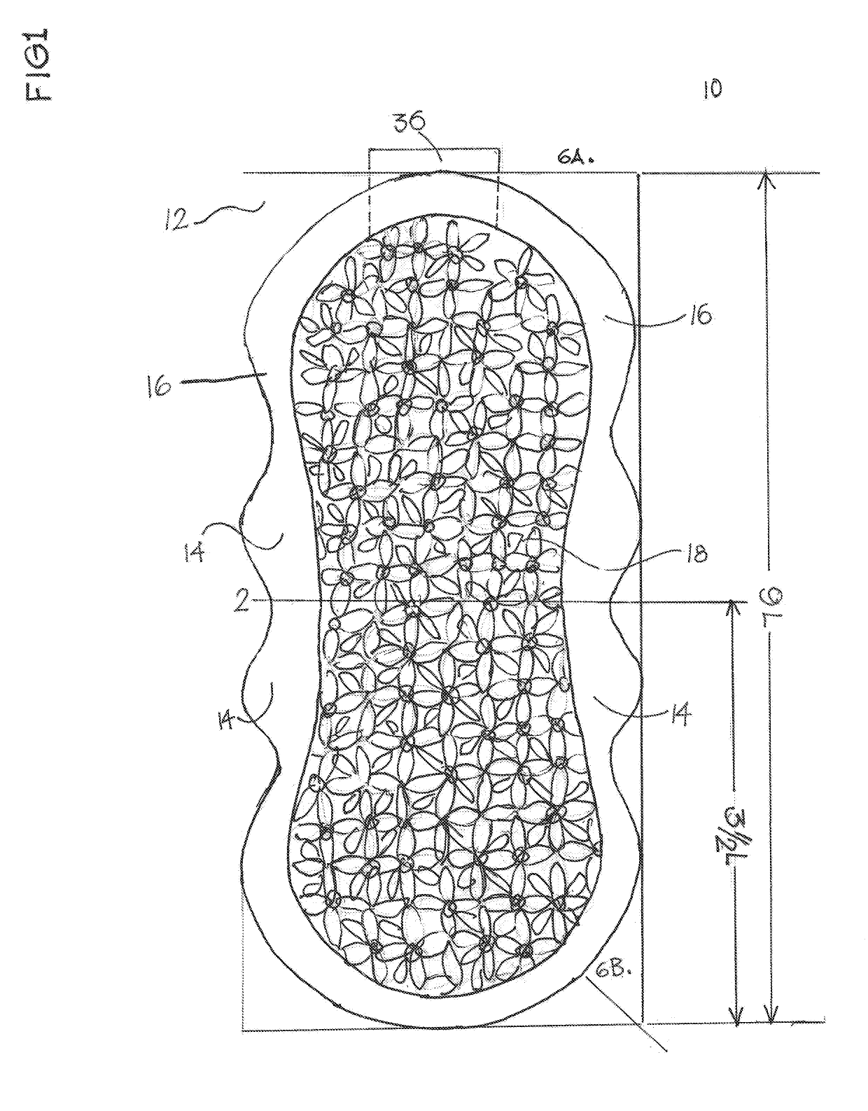

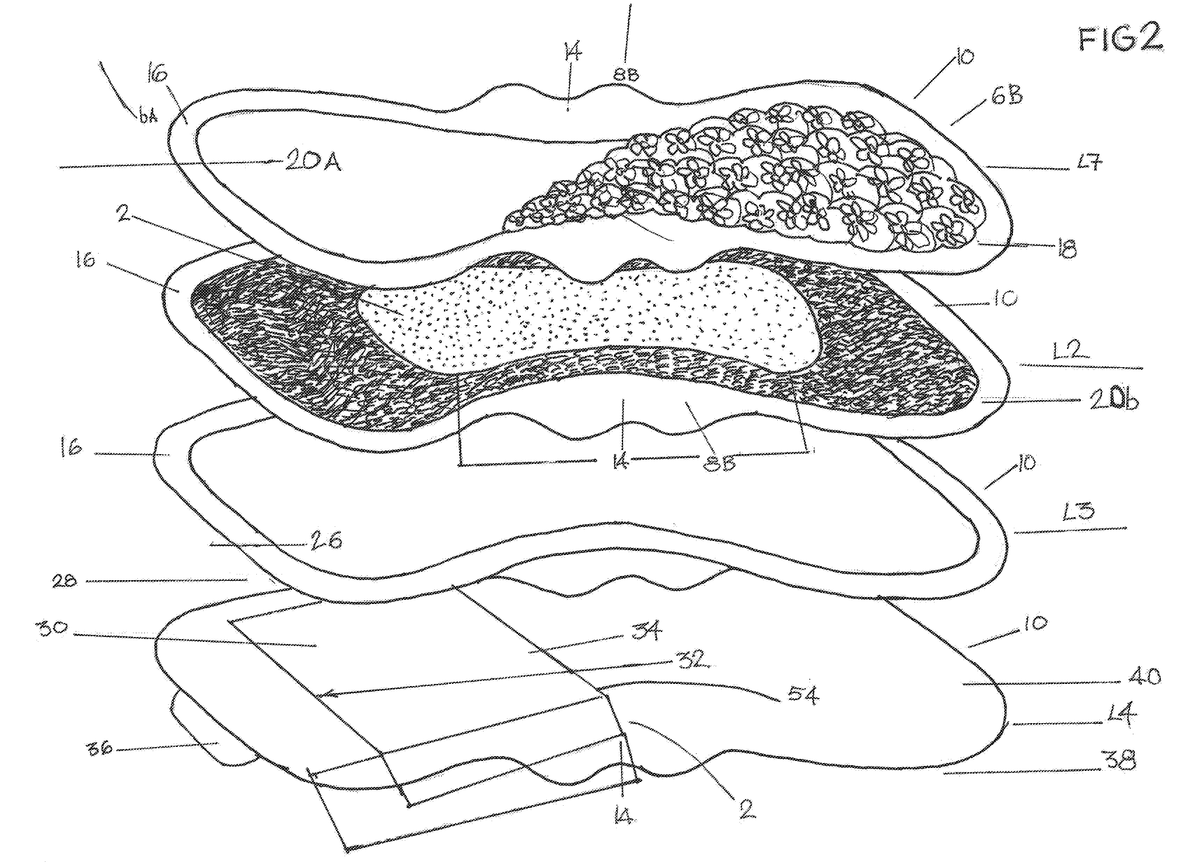

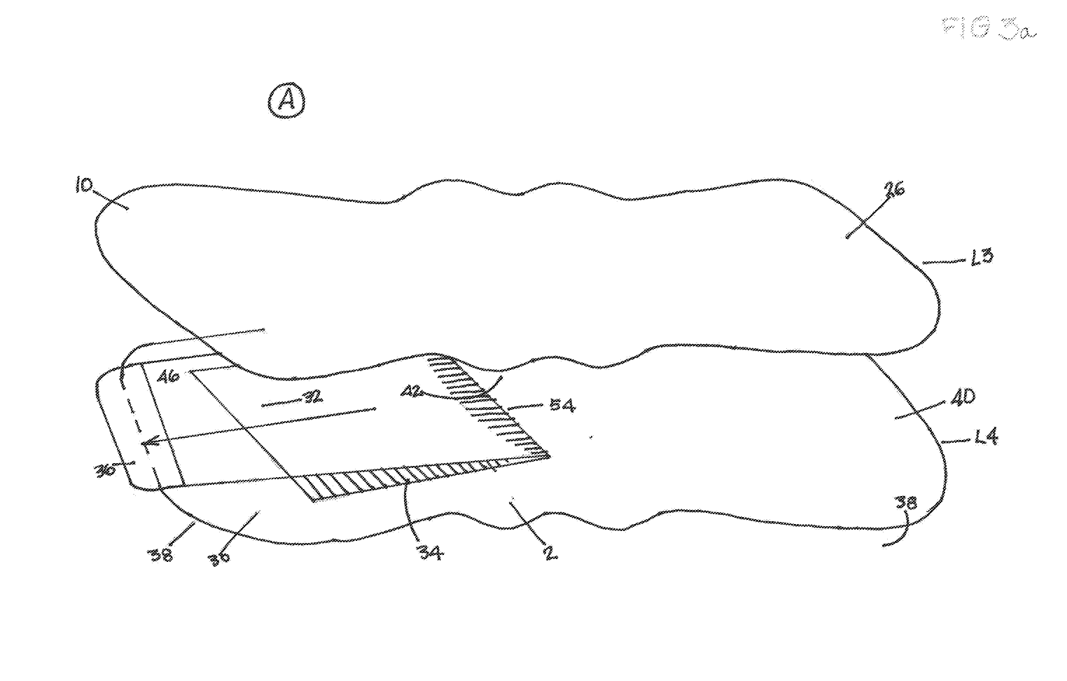

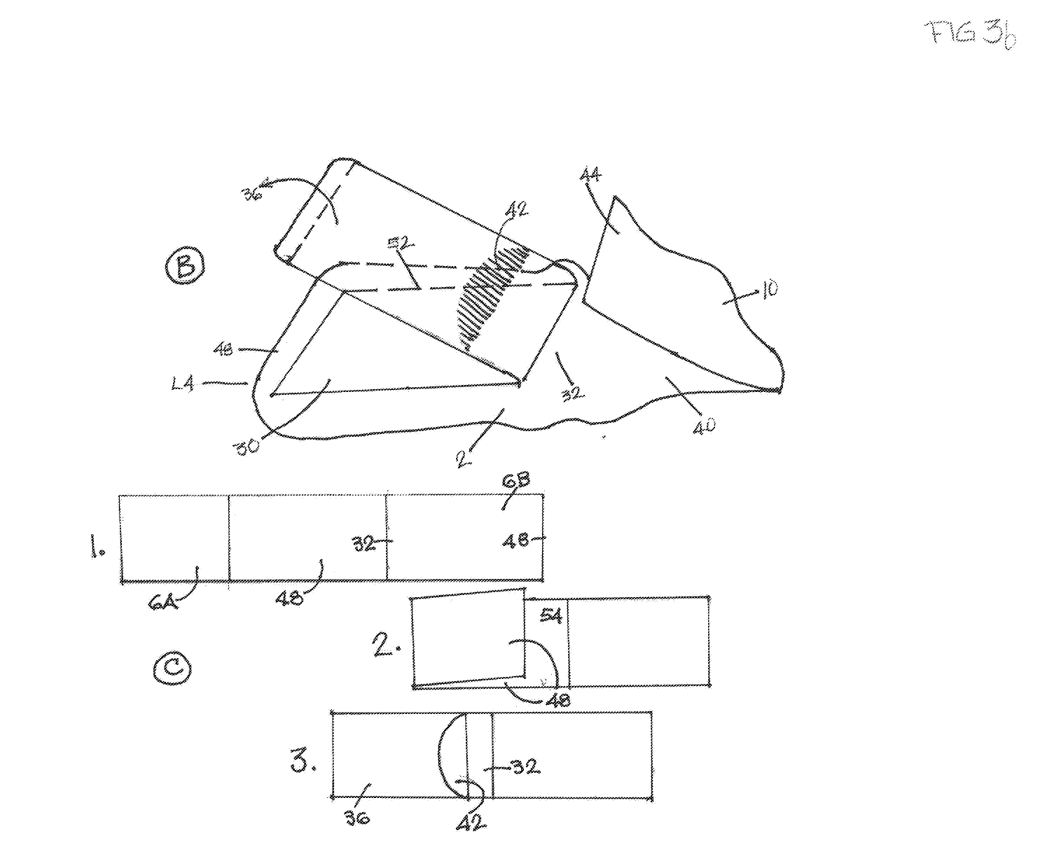

Components will contain:
A, B, C
1A,
1, 2, 3, 4, 5
FIGS. 1-5
Layers L1-4 (FIG. 2)
Steps 1-3 (FIG. 3)
   Each number will label its component below.
2—Center
6A—Front up/Top up
6B—Back up
8A—Left Side
8B—Right Side
10—Feminine Napkin/Absorbent Pad/Oval
12—Women/Female Undergarment Clothing
14—Secure Flaps/Left & Right
16—Extended Plastic Outer Layer
18—Flower Pattern/Air flow
20A—Absorbing Pad
20B—Absorbing Pad with Air flow Holes
22—Absorbing Material
26—Protective Barrier
28—Film to Seal Component together top/bottom
30—Integrated Plastic Bag
32—Base Film
34—Plastic Bag Adhere
36—Pull Flap forward to remove Bag
38—Bottom all around Adhesive
40—Material/Plastic Base Film
42—Opening of Bag
44—Releasing of Bag
48—Glue
52—Film pull Apart
54—Folding
56—Integrated Bag Extended out
58—Roll napkin toward bag/rolled
60A—Left Hand
60B—Right Hand
62A—Left Thumb tucking bag in
62B—Right Thumb
63A—Left Pointer Finger
63B—Right Pointer Finger tucking bag in

### DETAILED DESCRIPTION OF DRAWINGS

### FIG. 1.

Referring to the drawing, a feminine napkin and/or absorbent pad, oval shape 10, illustrate the finish product look with an extended pull flap 36, which will contain the invention of the integrated plastic bag. The feminine napkin and/or absorbent pad is (on a small unit) and front up 6A to back up 6B, in an open position in between an ideal feminine undergarment/clothing 12. The feminine napkin and/or absorbent pad 10, contains unfolded and elongated flaps 14 on left 8A side and right side 8B (small unit), to secure the undergarments/clothing 12, from moving while walking. Underneath the flaps 14 will contain adhesive 38 (not illustrated in the figure only describe). The feminine napkin and/or absorbent pad 10 contains an extended plastic outer layer 16, to prevent any additional bodily fluid from leaking onto the undergarments/clothing 12. The flower pattern 18, possesses an individual absorbent 20A and 20B, with airflow holes to circulate air and help keep the skin dry.

### FIG. 2.

Illustration of the feminine napkin 10 is shown with layers separated to describe each function of the components facing front up 6A to back up 6B.

Layer L1, illustrates the component left 8A and right 8B flaps 14, the extended plastic outer layer 16, the absorbing pad 20A and 20B. The image is shown with half flower patterns/airflow 18, and a solid non pattern side to illustrate two different components when separated, it will contain airflow holes 18, to hold moisture and keep skin dry (mini holes and lines pattern material).

Layer L2, illustrates a more detail design of the second absorbent pad 20b and a center point 2, for which the bodily fluid is expected to absorb the most, is illustrated with a focal point padding material 22, extended plastic outer layer 16 and secure flaps 14.

Layer L3, illustrates a protective barrier sheet 26, to capture additional bodily fluid to prevent the fluid from leaking through the feminine napkin 10 and extended plastic outer layer 16.

Layer L4, contains the integrated plastic bag 30, folded and attached 54, in the center 2 and adhere 34 to the feminine napkin 10 with glue (described but not appearing in layer 4 drawing). The base film 32, holds the bag in place with glue preventing the plastic bag 30 from extending out fully. The pull flap forward 36 is exposed and extended at the front 6A, tip of the feminine napkin 10. This will pull the integrated bag 30 out of the pad 10. The adhesive 38, is located on the bottom underneath the feminine napkin 10 to prevent moving while walking. The overall layer L4, material is plastic base film 40. Note, each component is sealed together with a film material/glue 28.

### FIG. 3.

Illustrates A and B of the feminine napkin and/or absorbent pad 10 components and C the integrated bag creation.

A. Layer L3 is the solid protective barrier 26 and Layer L4 illustrates the positioning of the plastic bag between both layers along with details of the functions.

B. Layer L4, contains the integrated plastic bag 30, centered 2, folded 54, front up 6A, and on a base film material 40 is with a removable base film 32. This will allow the plastic bag 30, to adhere 34, when pulled forward at the tip by the flap 36. The integrated bag contains an opening 42 for the feminine napkin 10 to roll into once fully extended (the extending of the bag is not shown in Layer L4). The release of the bag 44, will allow the bag to be pulled forward and away from the base film 32, the bag moves and the film on left and right side 8A and 8B pulls apart 52. The glue 48 is attached to secure the bag from exiting off the plastic base film 40.

C. consist of steps 1, 2, and 3 for creating the integrated plastic bag 30. Step 1. illustrates the front 6A of bag 30 with a base film 32 and glue 48. Step 2. Illustrates the bag 30 folding back 54, to be glued 48, creating an opening for the feminine napkin and/or absorbent pad 30 to be rolled into (Step 2 describes the feminine napkin to be rolled into, not Illustrated). Step 3 Illustrates the integrated plastic bag 30 folded back at the base film 32, with the pull flap at the front 6A of bag 30. The opening of the bag 42 (where the feminine napkin rolls into, is only describe not illustrated).

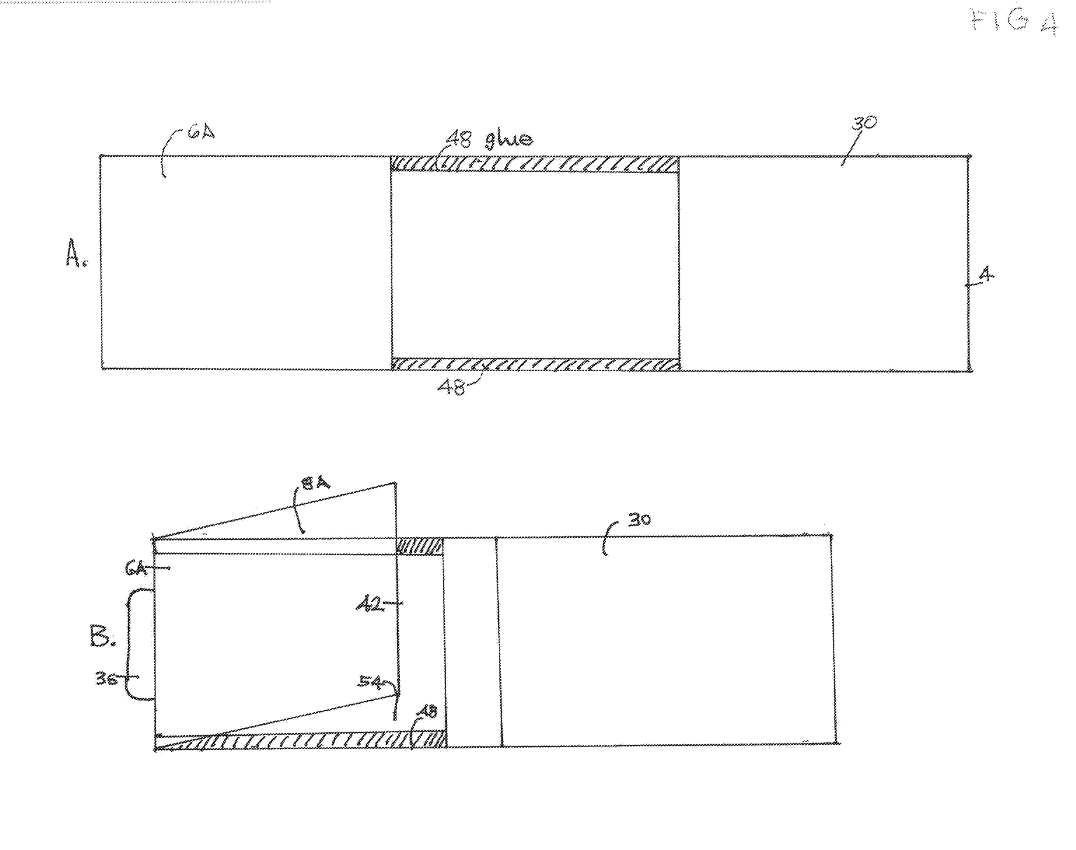

### FIG. 4.

The integrated plastic bag 30 is outlined using A and B Illustration.

A. illustrates the plastic bag 30, extended from front 6A to back 6B and the center 2, for glue 48 of the portion of the bag.

B. contains the integrated plastic bag 30, front top up 6A, folding 54 left 8A and right 8B sides back and down onto the plastic bag 30, gluing 48, the bag 30 together (onto the front plastic base film 40 not Illustrated on B. FIG. 4).

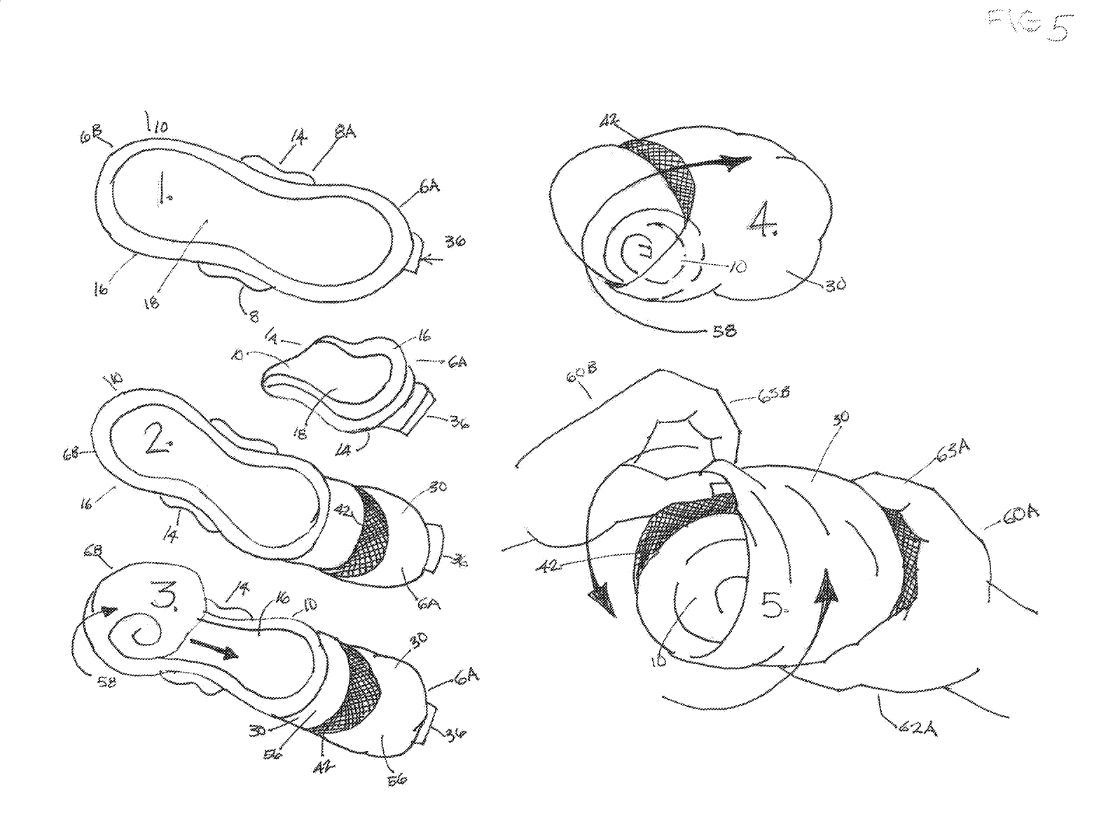

### FIG. 5.

The integrated plastic bag 30, is illustrated listing the numbers of each function from 1-5, 1A, start to finish.

Number 1. The feminine napkin and/or absorbent pad 10, appears face up, front 6A to back 6B, top layer L1 with secure extended flaps 14, on the left 8A and right side 8B. The feminine napkin 10, contains air flow holes 18 to keep skin dry and to protect undergarments extended plastic outer layer all around the pad 16. The pull flap forward is visible for gripping to remove bag 36.

Number 1A. Provides a half illustration of the feminine napkin and/or absorbent pad 10, facing front up/top up 6A, with part of the extended plastic layer 16, Air flow flower pattern 18, and the pull flap forward 36 with an arrow indicating to pull bag out.

Number 2. Illustrates the feminine napkin 10, front 6A back 6B, secured flaps 14, extended outer layer 16, and Flower pattern air flow holes 18, with the integrated plastic bag 30 pulled forward and extended out 56, by its flap 36, visually seeing the opening of the bag 42.

Number 3. Illustrates, the feminine napkin and/or absorbent pad 10, the secured flaps 14, and the integrated plastic bag 30. From the back 6B is seen rolling napkin 58 toward the front 6A into the opening of the bag 42 after the pull flap 36 extends the bag 56. (The arrows indicate to roll pad forward).

Number 4. The feminine napkin and/or absorbent pad 10, is rolled 58 into the opening 42 of integrated plastic bag 30.

Number 5. Illustration shows the feminine napkin and/or absorbent pad 10, rolled into the opening 42 of the integrated plastic bag 30. To discreetly dispose, block odor, leakage, and exposure of the feminine napkin and/or absorbent pad 10 both hands are seen tucking the content into the integrated plastic bag 30, by using two hands 60A left and 60B right, left 62A, right 62B thumb, along with left pointer finger 63A and right 63B pointer finger.

What is claimed is:

1. A disposable integrated plastic bag with a pull flap at the top tip combined between the 3^{rd} and 4^{th} layers of a related feminine sanitary napkin/absorbent pad. The pull flap releases the integrated plastic hag and extends mid-way out between the layers and opens with the inside or entry of the bag facing the user. The technique of the integrated bag allows the soiled pad to be rolled/wrapped into the bag without contamination on the hands, eliminating odor, exposure, and excessive use of toilet tissue. The feminine napkin/absorbent pad is rolled/wrapped down in the bag to discreetly dispose the proper way. The integrated plastic bag is light weight, thin, with colors and/or pattern. The feminine napkin/absorbent pad is integrated with the disposable plastic bag as one unit, packaged with different positive words on the wrapper prior to using.
